(19) United States
(12) Patent Application Publication
(10) Pub. No.: US 2010/0189595 A1
(43) Pub. Date: Jul. 29, 2010

(54) Method of Controlling Organoleptic Odors

(75) Inventor: Joseph R. Webster, Charlotte, NC (US)

Correspondence Address: The Hanf Law Firm PLLC;7845 Colony Road, Suite C4, Box 158, Charlotte, NC, 28226, US

(73) Assignee: Lehigh Tecghnologies Inc., Naples, FL (US)

(21) Appl. No.: 12/361,873

(22) Filed: Jan. 29, 2009

(51) Int. Cl. B01J 29/04; A61L 9/014

(52) U.S. Cl. 422/4; 502/62

(57) ABSTRACT

A method is taught for capturing organoleptic odor. Where a functional additive has odors from a plurality of organolepic sources, and is blended with an odor control agent and a resin to produce a blend, where the blend exhibits at least a 5% reduction in odor based on a standardized odor test SAE-J1351. The odor control agent is selected from the group but not limited to: nepheline syenite, silica gel, hydrogels, hard and soft clays, bentonite, clinoptilolite, hectorite, cationic exchanged clinoptilolites, cerium, cesium, chabazite, faujasite, gmelinite, brewsterite, calcium silicate, hydrotalcites, zinc or magnesium aluminum hydroxy carbonates, zinc oxide, zinc hydroxide, zinc carbonate, calcium oxide, calcium hydroxide, calcium carbonate, potassium meta phosphate, silver oxide, magnesium hydroxide, magnesium oxide, copper oxide, ferric and ferrous oxides, sorbitol, glucitol, mannitol, glucose, dextrose, dextrin, allophanes, silica, sodalite, silicon oxide, aluminum oxide, natural zeolites, manganese dioxide, nano zinc oxide and nano titanium and combination thereof.

### BACKGROUND OF THE INVENTION

The present invention provides a method for capturing organoleptic odor in a functional additive, where this functional additive is fraught with odors from a plurality of organolepic sources.

Used tires from motor vehicles are one of the largest and most problematic sources of waste, due to the large volume produced and their durability. It has been estimated that one tire is discarded per person per year. The U.S. Environmental Protection Agency reports over 290 million scrap tires were generated in 2003. Of the 290 million, 45 million of these scrap tires were used to make automotive and truck tire re-treads. The tire industry does use a small percentage of this waste in the production of new tires, however due to safety issues, the tire industry's recycled rubber content can only be 5-15%, new tires must be manufactured primarily from virgin rubber. This leaves the majority of these tires to be disposed of.

Waste tires are not a good candidate for landfills, due to their large volumes and 75% void space, which quickly consume valuable space. Tires can trap methane gases, causing them to become buoyant, or ‘bubble’ to the surface. This ‘bubbling’ effect can damage landfill liners that have been installed to help keep landfill contaminants from polluting local surface and ground water. This has resulted in landfills minimizing their acceptance of whole tires. The other alternative is stockpiling these waste tires, unfortunately waste tire stockpiles create a great health and safety risk. Tire fires can occur easily, burn for months, create substantial pollution in the air and ground, and result in the site becoming a Superfund cleanup site. Another health risk associated with waste tires is that tire piles provide harborage for vermin and a breeding ground for mosquitoes that may carry diseases. In 2004 the number of waste tires in storage in the United States was estimated to be around 275 million. Currently there is a great need to find creative recycling opportunities for these waste tires.

There have been several uses for this recycled tire waste. One use has been to shred these tires into chunks, and us them as a filler for paving products. Another use has been to burn tire chips in industrial boilers or incinerators. A more significant break through in the technology of recycling tires has been introduced by Lehigh Technologies, Inc., Naples, Fla., which has come up with several patented processes (U.S. Pat. Nos. 7,108,207 and 7,093,781) for recycling tires by cryogenically grinding the tires into a fine material, which is more suitable for use in wide variety of rubber, plastics and other applications.

### SUMMARY OF THE INVENTION

A method for capturing organoleptic odor in a blend of a functional additive with a resin. The functional additive has odors from a plurality of organolepic sources, and is blended with an additive, and a resin to produce a blended resin, where the blended resin exhibits at least a 5% reduction in odor based on a standardized odor test SAE-J1351. The additive is selected from the group, but not limited to: nepheline syenite, silica gel, hydrogels, hard and soft clays, bentonite, clinoptilolite, hectorite, cationic exchanged clinoptilolites of zinc, silver,copper, ammonia, acid functionality,lithium,platinum, gallium, cerium, cesium. Chabazite, faujasite, grnelinite, brewsterite, calcium silicate, magnesium aluminum hydroxy carbonates, zinc oxide, zinc hydroxide, zinc carbonate, calcium oxide, calcium hydroxide, calcium carbonate, potassium meta phosphate, silver oxide, magnesium hydroxide, magnesium oxide, copper oxide, ferric and ferrous oxides, sorbitol, glucitol, mannitol, glucose, dextrose, dextrin, allophanes, silica, sodalite, silicon oxide, aluminum oxide, natural zeolites, manganese dioxide, nano zinc oxide and nano titanium and combination thereof.

### DETAILED DESCRIPTION OF THE INVENTION

One way to help with the problem of unwanted waste tires is to grind them up and use them as a functional additive with a resin in order to make an injection molded product. One of the problems with this approach is that due to the complex make up of tire rubber it may have an odor from a plurality of organoleptic sources which would make injection molded pieces using this recycled rubber less attractive than parts mad with a virgin plastic.

Surprisingly this problem can be mediated by a process of blending a resin with a functional additive where the functional additive is added at a level from 0% to 70% by weight of the resin. Then an additive is added at a level from 1% to 35% based on the weight of the resin, this additive surprisingly reduces odor, while it is not know how this is accomplished, it is believed that the addition of this additive reduces the level of volatile and semi-volatile organic compounds that come off the mixture of the resin and the functional additive. It has been found that levels of functional additive having a lower end of 3, 5, 7, 10, 15 and 20 percent and an upper end of 60, 55, 50, 45 and 40 percent work well. It has been found that levels of the additive having a lower end of 2, 3, 5, 7, 8, 10 percent and an upper end of 30, 27, 25, 23 and 20 percent work well.

The resins which can be used with this invention are quite varied and include, but are not limited to: polypropylene (PP), as a homopolymer or a copolymer as well as syndiotactic PP; polyethylene (PE), including high density PE (HDPE), low density PE (LDPE), LLDPE and Ultra High Molecular Weight PE (UHMW) PE; condensation polymers such as Nylon, Polybutylene Terephthalate (PBT), polycarbonate (PC); additional polymers such as polystyrene, High Impact Polystyrene (HIPS), Poly(Styrene Acrylonitirle) (SAN), Poly(Acrylonitrile Butadiene Styrene)(ABS) and Poly(acrylic Styrene Acrylonitrile) ASA; tackified resins and hot melts such as Poly(Styrene Butadiene Styrene) (SBS); Poly(Styrene Ethylene Butadiene Styrene) SEBS; polybutylene; cured and uncured EPDM rubbers; acetal; acrylic; phenylene oxide; polyester; polysulfone; urethane; polyurethanes; vinyl and combinations thereof.

The functional additive can be derived from a recycled source and can include plastic, rubber and tires. An example of a functional additive is PolyDyne™ available from Lehigh Technologies, Inc., Naples, Fla. and is made from recycled tires. Lehigh Technologies, Inc. has several patented processes, U.S. Pat. Nos. 7,108,207 and 7,093,781, for recycling tires by cryogenically grinding the tires into a fine material that is suitable for use in wide variety of applications. As used in this application the term functional additive includes: additives, filler modifiers, fillers, reinforcement modifiers, polymer modifiers and the like.

The additive, which will be referred to as the odor control agent, is selected from the group including: nepheline syenite, nepheline, silica gel, hydrogels, hard and soft clays, purified and unpurified hydrous magnesiuim aluminum silicates, bentonite, clinoptilolite (Bulgarian) from both sodium and potassium clinoptilolite forms, hectorite, cationic exchanged clinoptilolites (with silver, copper, nickel), cerium, cesium and other cation elements exchanged within the cage structure, chabazite, faujasite, gmelinite, brewsterite, calcium meta silicate, calcium silicate, magnesium aluminum hydroxy carbonates, zinc oxide, zinc hydroxide, zinc carbonate, calcium oxide, calcium hydroxide, calcium carbonate, potassium meta phosphate, silver oxide, magnesium hydroxide, magnesium oxide, copper oxide, ferric and ferrous oxides, sorbitol, glucitol, mannitol, glucose, dextrose, dextrin, allophanes, structured silica, amorphous silicas, sodalite, silicon oxide and dioxide, aluminum oxide and dioxides, natural zeolites, synthetic zeolites, ammonia treated and acid treated clinoptilolite, manganese dioxide, metallocene waxes, nano zinc oxide and nano titanium and combination thereof.

The term organoleptic as used in this application means an odor, which comes from a chemical or, that is derived from petroleum refining. It is also noted that in certain applications, the raw materials are often exposed to pressure, residence time and temperature during processing. In this way, ingredients present in the raw materials could react when subjected to these high temperatures and pressures, and give off chemicals or compounds not otherwise present in the raw materials. The high temperatures and pressures could be the result of injection molding this composition. These resulting chemicals and compounds might also produce organoleptic odors.

The addition of the odor control agent reduces the level of volatile and semi-volatile organic compounds that come off the mixture of the resin and the functional additive. These compounds are commonly referred to VOC (volatile organic compounds) and HAPS (Hazardous Airborne Pollutants). This results in a reduction of odor as measured by a standardized odor test SAE-J1351 where a panel of people rate the subjective odor of a heated sample and rank the odor based on the odor wheel from the test. In one embodiment of the invention the addition of the odor control agent resulted in at least a 5% reduction in odor based on a standardized odor test SAE-J1351. In another embodiment the improvement was at least 10%. This standardized odor test was developed by the Society of Automotive Engineers and is titled “Hot Odor Test For Insulation Materials” designated as SAE-J1351.

In one of the embodiment of the invention the resin used has a melting point from 100 to 280° C. It has been found that resins falling in the range of 115 to 250° C. work well.

In an embodiment of the invention the odor control agent is a blend of two or more odor control agents selected from the group of: nepheline syenite, nepheline, silica gel, clinoptilolite, natural zeolite and synthetic zeolite. In one of the embodiments of the invention the two odor control agents are added in a ratio of 5:1 to 1:5.

This method for chemically capturing organoleptic odor in one embodiment, has the functional additive added at a level of from 5% to 45% by weight of the plastic resin and the level of the odor control agent is from 5% to 25% by weight of the plastic resin.

In one of the embodiments of the invention the base resin may be from a recycled source or it may have a compound added to it that would give it an unpleasant odor in such a case the additive or odor control agent might be added directly to the plastic resin without the use of any functional additive to remediate any unpleasant odor that the resin might have.

In an embodiment of the invention the odor control agent is a blend of a magnesium aluminum silicate with an odor control agents selected from the group of: nepheline syenite, nepheline, silica gel, clinoptilolite, natural zeolite and synthetic zeolite. An example of the magnesium aluminum silicate is a magnesium aluminum phyllosilicate such as Palygorskite or Attapulgite. Palygorskite or Attapulgite is a magnesium aluminum phyllosilicate having the formula (Mg,Al)₂Si₄O₁₀(OH).4(H₂O) which occurs in clay soil in the southeastern part of the United States. It is sometimes referred to as fuller's earth. This material is more thermally stable at temperatures above 200 degrees C., it is believed that they are more surface active and perform better than soft or hard Kaolin or bentonite clays.

### EXAMPLES

A study was done with a black pigmented 90 melt flow (190C, 2.16 kg) face cut polyethylene blended with Polydyne 80, a 80 mesh 180 micron or 0.0070 inch particle size cryogenic recycled tire rubber, and melt compounded on a 21 mm twin screw extruder. Over 80 formulations were melt compounded and dried with conventional approaches and placed into aluminized Mylar zip lock bags for storage. Three weeks later samples were tested for odor via a SAE J1351 and a four-member odor panel, of non-smokers, were assembled. The results of a two day odor panel study of different loads of Polydyne rubber samples clearly illustrated a significant reduction in post melt compounded odor of pellets heated for one hour at 65C in a circulating air oven.

TABLE A

| Column1 | [Alterin 110] | Additive | Level |
|---|---|---|---|
| 0 | 0 | | |
| 1 | 5 | PCA1 | 0.5 |
| 2 | 5 | PCA3 | 0.5 |
| 3 | 5 | PCA4 | 0.5 |
| 4 | 5 | PCA5 | 0.5 |
| 5 | 5 | PCA7 | 0.5 |
| 6 | 5 | PCA8 | 0.5 |
| 7 | 5 | PCA10 | 0.5 |
| 8 | 5 | PCA11 | 0.5 |
| 9 | 5 | PCA12 | 0.5 |
| 10 | 5 | PCA15 | 0.5 |
| 11 | 5 | PCA1 | 1 |
| 12 | 5 | PCA3 | 1 |
| 13 | 5 | PCA4 | 1 |
| 14 | 5 | PCA5 | 1 |
| 15 | 5 | PCA7 | 1 |
| 16 | 5 | PCA8 | 1 |
| 17 | 5 | PCA10 | 1 |
| 18 | 5 | PCA11 | 1 |
| 19 | 5 | PCA12 | 1 |
| 20 | 5 | PCA15 | 1 |
| 21 | 5 | PCA16 | 1 |
| 22 | 5 | PCA17 | 1 |
| 23 | 5 | PCA18 | 1 |
| 24 | 5 | PCA24 | 1 |
| 25 | 5 | PCA20 | 1 |
| 26 | 5 | PCA21 | 1 |
| 27 | 5 | PCA22 | 1 |
| 28 | 5 | PCA23 | 1 |
| 29 | 5 | PCA19 | 1 |
| 30 | 5 | PCA25 | 1 |
| 31 | 5 | PCA1 | 1 |

TABLE B

| | [Alterin 110] | [Polydyne 80] |
|---|---|---|
| 31B | 5 | 10 |
| 32 | 5 | 20 |
| 33 | 5 | 30 |
| 34 | 5 | 40 |
| 35 | 5 | 50 |

TABLE C

| | [EG600] | [Polydyne 80] |
|---|---|---|
| 36 | 5 | 10 |
| 37 | 5 | 20 |
| 38 | 5 | 30 |
| 39 | 5 | 40 |

The EG 600 30% masterbatch of Alterin 110 in a polyethylene carrier gives final level of Alterin 110 at 5% by weight level.

TABLE D

| | [Alterin 110] | [Polydyne] | [Celspan 610] |
|---|---|---|---|
| 41 | 5 | 10 | 2 |
| 42 | 5 | 20 | 2 |
| 43 | 5 | 30 | 2 |
| 44 | 5 | 40 | 2 |
| 45 | Eliminated | | |

Celspan 610 is a proprietary molecular modifier of Phoenix Plastics.

TABLE E

| | [Alterin 110] | [Polydyne 80] |
|---|---|---|
| 46 | 0 | 20 |
| 47 | 3 | 20 |
| 48 | 5 | 20 |
| 49 | 7 | 20 |
| 50 | 10 | 20 |

TABLE F

| | [Atlerin 110] | [Polydyne 80] |
|---|---|---|
| 51 | 0 | 40 |
| 52 | 3 | 40 |
| 53 | 5 | 40 |
| 54 | 7 | 40 |
| 55 | 10 | 40 |

TABLE G

| | [Alterin 110] | [PCA10] | [Polydyne] |
|---|---|---|---|
| 56 | 0 | 5 | 20 |
| 57 | 3 | 2 | 20 |
| 58 | 5 | 5 | 20 |
| 59 | 7 | 3 | 20 |

TABLE H
60, 61, 62, 63 Eliminated

| | [Alterin 110] | [PCA3] | [poly 80] |
|---|---|---|---|
| 64 | 0 | 10 | 20 |
| 64B | 0 | 5 | 20 |
| 65 | Eliminated | | |
| 66 | 5 | 5 | 20 |
| 67 | 2.5 | 2.5 | 20 |

TABLE I

| | [Alterin 110} | [PCA4] | [poly80] |
|---|---|---|---|
| 68 | 0 | 10 | 20 |
| 68B | 0 | 5 | 20 |
| 69 | Eliminated | | |
| 70 | 2.5 | 2.5 | 20 |
| 71 | Eliminated | | |

TABLE J
72 through 87 eliminated
16 samples total
Acid versus Alkaline Environment

| | [Alterin 110] | [PCA7] | [poly80] |
|---|---|---|---|
| 88 | Eliminated | | |
| 89 | Eliminated | | |
| 90 | 0 | 5 | 20 |
| 91 | Eliminated | | |
| 92 | Eliminated | | |
| 93 | Eliminated | | |
| 94 | 5 | 5 | 40 |
| 95 | Eliminated | | |

TABLE K
Spiking of Alterin 110

| | [Alterin 110] | [PCA5] | [Poly80] |
|---|---|---|---|
| 96 | 0 | 10 | 40 |
| 97 | Eliminated | | |
| 98 | 5 | 5 | 40 |
| 99 | Eliminated | | |

TABLE L

| | [Alterin 110] | [PCA2] | [Poly80] |
|---|---|---|---|
| 100 | 0 | 10 | 20 |
| 101 | 3 | 7 | 20 |
| 102 | 5 | 5 | 20 |
| 103 | 7 | 3 | 20 |
| 104 | Eliminated | | |
| 105 | Eliminated | | |
| 106 | Eliminated | | |
| 107 | Eliminated | | |

TABLE M

| | [poly 80] | [Alterin 110] | PCA20 | PCA18 | PCA25 | PCA8 |
|---|---|---|---|---|---|---|
| 108 | 20 | 5 | 1 | | | |
| 109 | 20 | 5 | | 1 | | |
| 110 | 20 | 5 | | | 1 | |
| 111 | 20 | 2.5 | | | | 2.5 |
| 112 | 20 | 5 | | 0.5 | 0.5 | |

TABLE N
113 to 115 Eliminated 3 total

| | [Alterin 110] | [PCA19] | [poly80] |
|---|---|---|---|
| 116 | 5 | 5 | 40 |
| 117 | 0 | 10 | 40 |
| 118 | Eliminated | | |
| 119 | Eliminated | | |

Total of 80 Samples for Melt Extrusion, where the additives consisted of the following:

TABLE O

| | |
|---|---|
| PCA1: | Copper Oxide Black |
| PCA2: | DHT-4A |
| PCA3: | DIXIE CLAY |
| PCA4: | MCNAMAEE CLAY |
| PCA5: | ZEODEX ZnCl2 occluded (2/6/01) |
| PCA6: | HTZ CaCO3 (307-1) |
| PCA7: | Zeodex Hydrogen (HCl) |
| PCA8: | Zeodex NH4 treated |
| PCA9: | Zeodex Alterin 110 |
| PCA10: | Gascil 23D structured silica |
| PCA11: | Unimin Miniblaock HC400 |
| PCA12: | Angula Silica Promi De Occidente |
| PCA13: | Celspan OE MB 32% EG600 |
| PCA14: | Celspan 610 |
| PCA15: | Advera 401 lot 401-009-01 PQ Corp. |
| PCA16: | Kadox 911 |
| PCA17: | Kadox 911C |
| PCA18: | Silver oxide |
| PCA19: | Charcoal |
| PCA20: | Activated Charcoal Alltech Associates |
| PCA21: | Iron oxide black |
| PCA22: | Manganese dioxide |
| PCA23: | 10% platinum on charcoal |
| PCA24: | zinc powdered metal. |
| PCA25: | Nano Zinc oxide (Elementis) |

TABLE P

| | | | |
|---|---|---|---|
| | Polydyne 80 | Observations | Intensity Rating |
| Designation | Level | After 65 C./1 hour | Average (4) |

| | | | | |
|---|---|---|---|---|
| 1 | Control | None | No Moisture | 1.825 |
| | Base resin | | | |
| 2 | 46 | 20% | No Moisture | 2.625 |
| 3 | 47 | 20% | Lots of Moisture | 2.425 |
| 4 | 48 | 20% | Lots of Moisture | 2.25 |
| 5 | 49 | 20% | Lots of Moisture | 2 |
| 6 | 50 | 20% | Lots of Moisture | 1.825 |
| 7 | 64 | 20% | No Moisture | 1.3 |
| 8 | 64B | 20% | No Moisture | 1.275 |
| 9 | 66 | 20% | No Moisture | 1.25 |
| 10 | 67 | 20% | No Moisture | 1.875 |
| 11 | 68B | 20% | No Moisture | 2.5 |
| 12 | 68 | 20% | No Moisture | 1.6 |
| 13 | 70 | 20% | No Moisture | 1.75 |
| 14 | 100 | 20% | Sl.to No Moisture | 1.8 |
| 15 | 101 | 20% | Sl.to No Moisture | 1.65 |
| 16 | 102 | 20% | Sl.to No Moisture | 2.25 |
| 17 | 103 | 20% | High Moisture | 2.125 |
| 18 | 56 | 20% | High Moisture | 2.425 |
| 19 | 57 | 20% | High Moisture | 2.2 |
| 20 | 58 | 20% | High Moisture | 1.625 |
| 21 | 59 | 20% | High Moisture | 2.2 |
| 22 | 36 | 10% | High Moisture | 1.5 |
| 23 | 37 | 20% | High Moisture | 1.75 |
| 24 | 38 | 30% | High Moisture | 2.5 |
| 25 | 39 | 40% | High Moisture | 2.375 |
| 26 | 41 | 10% | High Moisture | 3.125 |
| 27 | 42 | 20% | High Moisture | 3.2 |
| 28 | 43 | 30% | High Moisture | 3.375 |
| 29 | 44 | 40% | High Moisture | 3.25 |
| 30 | 108 | 20% | Moisture | 3.375 |
| 31 | 109 | 20% | Moisture | 3.5 |
| 32 | 110 | 20% | Moisture | 3.375 |
| 33 | 111 | 20% | Moisture | 4.05 |
| 34 | 112 | 20% | Moisture | 3.375 |
| 35 | 51 | 40% | Moisture | 3.1 |
| 36 | 52 | 40% | Moisture | 2.75 |
| 37 | 53 | 40% | Moisture | 2.25 |
| 38 | 54 | 40% | Moisture | 2.625 |
| 39 | 55 | 40% | Moisture | 2.45 |
| 40 | 96 | 40% | Moisture | 4.125 |
| 41 | 98 | 40% | Moisture | 3.35 |

TABLE Q

| | | Polydyne 80 | Observations | Intensity Rating |
|---|---|---|---|---|
| | Designation | Level | After 65 C./1 hour | Average (4) |
| 42 | 31B | 10% | No Moisture | 2.125 |
| 43 | 32 | 20% | No Moisture | 2.375 |
| 44 | 33 | 30% | High Moisture | 2.375 |
| 45 | 34 | 40% | High Moisture | 2.125 |
| 46 | 35 | 50% | High Moisture | 2.5 |
| 47 | 90 | 20% | High Moisture | 2.375 |
| 48 | 84 | 40% | High Moisture | 1.875 |
| 49 | 116 | 40% | High Moisture | 2.487 |
| 50 | 117 | 40% | High Moisture | 2.25 |
| 51 | 0 | 40% | High Moisture | 2.5125 |
| 52 | 1 | 40% | High Moisture | 2.3 |
| 53 | 2 | 40% | High Moisture | 2.2 |
| 54 | 3 | 40% | High Moisture | 2.125 |
| 55 | 4 | 40% | High Moisture | 1.875 |
| 56 | 5 | 40% | High Moisture | 1.687 |
| 57 | 6 | 40% | High Moisture | 2.125 |
| 58 | 7 | 40% | High Moisture | 1.925 |
| 59 | 8 | 40% | High Moisture | 1.313 |
| 60 | 9 | 40% | High Moisture | 1.625 |
| 61 | 10 | 40% | High Moisture | 1.675 |
| 62 | 11 | 40% | High Moisture | 2.9625 |
| 63 | 12 | 40% | High Moisture | 2.625 |
| 64 | 13 | 40% | High Moisture | 2.5 |
| 65 | 14 | 40% | High Moisture | 2.175 |
| 66 | 15 | 40% | High Moisture | 2 |
| 67 | 16 | 40% | High Moisture | 2.625 |
| 68 | 17 | 40% | High Moisture | 2.375 |
| 69 | 18 | 40% | High Moisture | 1.75 |
| 70 | 19 | 40% | High Moisture | 2.75 |
| 71 | 20 | 40% | High Moisture | 1.75 |
| 72 | 21 | 40% | High Moisture | 1.925 |
| 73 | 22 | 40% | High Moisture | 1.8 |
| 74 | 23 | 40% | High Moisture | 3 |
| 75 | 24 | 40% | High Moisture | 2.125 |
| 76 | 25 | 40% | High Moisture | 1.75 |
| 77 | 26 | 40% | High Moisture | 1.875 |
| 78 | 27 | 40% | High Moisture | 1.75 |
| 79 | 28 | 40% | High Moisture | 2.37 |
| 80 | 29 | 40% | High Moisture | 1.875 |
| 81 | 30 | 40% | High Moisture | 2 |
| 82 | 31 | 40% | High Moisture | 2.125 |

The results of a two day odor panel study of 20% and 40% loaded Polydyne rubber samples clearly illustrated a significant reduction in post melt compounded odor of pellets heated for one hour at 65C in a circulating air oven. The post melt compounded pellets were stored for three weeks in the dark and then conditioned for 24 hours at room temperature in 32 ounce pre-conditioned glass jars and then placed in a oven at 65C for one hour. Three male and one female who reportedly had a very sensitive nose were chosen for a subjective odor panel. All were non-smokers and educated in the sciences.

The average of four odor intensity rankings based on SAE J1351 procedures from 1 to 5.5 were provided each sample with additional data on perception of the type of odor being sensed. A target rating of below 2 was the goal. An odor intensity of 2 means slight but noticeable odor. A smell someone could easily ignore or that could be overpowered by some other smell. Ratings of 3 were described as definite odor, but not strong enough to be offensive, a smell that someone could become desensitized to over time.

It was noted early during initial melt extrusions that the “throw” or intensity of the odor coming off the Polydyne rubber added to the carrier resin was intense. The so-called throw of the odor is relevant because this is the odor the customer senses when adding the recycled rubber to a carrier resin. In addition the perception of what the final product will smell during storage. However, from our findings it is key to an appreciation of the final numbers from our odor panel on overall perception of odor. Since we cannot run an odor panel during melt extrusion we can only report on the subjective odor intensity and type as a comparison between initial melt extrusion and post melt extrusion. It is now our belief that the “throw” odor from Polydyne 80 is worse than the odor in post melt extrusion. The “throw” odor during melt extrusion is a period when all active chemical species from the cryogenic grinding are forced to the surface of the rubber and are actively flashed off during melt compounding giving a more intense odor. It is also a period when active radicals on the surface of the rubber due to melt compounding in a polymer matrix are more active and will cause side reactions during melt compounding e.g. crosslinking and degradation.

In the next test a black pigmented 90 melt flow (190C, 2.16 kg) face cut and recycled polyethylene was compounded with Polydyne 80 (a 80 mesh 180 micron or 0.0070 inch particle size cryogenic recycled tire rubber) and blended on a 21 mm twin screw extruder. In excess of 80 formulations were melt compounded and dried with conventional approaches and placed into aluminized Mylar zip lock bags for storage. Three weeks later samples were tested for odor via a SAE J1351 and a five-member odor panel of non-smokers and a six-member panel of non smokers. The results of these odor panel study of different loads of Polydyne rubber samples clearly illustrated a significant reduction in post melt compounded odor of pellets heated for one hour at 65C in a circulating air oven.

TABLE R

| | | | | | SAE J1351 | SAE J1351 |
|---|---|---|---|---|---|---|
| | | | | | Odor | Odor |
| | Polydyne | | | | Rating | Rating |
| Formulation: | 80 | Alterin 110 | % PCA | PCA | 5 members | 6 members |
| 1 | None | None | None | None | 2.1 | 2.08 |
| 2 | 20% | None | None | None | 3.2 | 3.25 |
| 3 | 20% | 3% | None | None | 2.5 | 2.58 |
| 4 | 20% | 5% | None | None | 2.5 | 2.5 |
| 5 | 20% | 1.5 | 1.50% | Dixie Clay | 2.4 | 2.25 |
| 6 | 20% | 2.50% | 2.50% | Dixie Clay | 2.3 | 2.166 |
| 7 | 20% | 1.50% | 1.50% | McNamee | 2.5 | 2.33 |
| | | | | Clay | | |
| 8 | 20% | 2.50% | 2.50% | McNamee | 2.1 | 2 |
| | | | | Clay | | |
| 9 | 20% | 1.50% | 1.50% | HC400 | 2.5 | 2.33 |
| 10 | 20% | 2.50% | 2.50% | HC400 | 2.5 | 2.25 |
| 11 | 20% | 1.50% | 1.50% | HC1400 | 2.4 | 2.166 |
| 12 | 20% | 2.50% | 2.50% | HC1400 | 2 | 1.833 |
| 13 | 20% | 1.50% | 1.50% | HC2000 | 2.7 | 2.5 |
| 14 | 20% | 2.50% | 2.50% | HC2000 | 2.3 | 2.166 |
| 15 | 20% | 1.50% | 1.50% | HC2100 | 2.3 | 2.166 |
| 16 | 20% | 2.50% | 2.50% | HC2100 | 1.6 | 1.583 |
| 17 | 20% | | 3% | Smelinite | 1.9 | 1.75 |
| 18 | 20% | | 6% | Smelinite | 1.6 | 1.5 |
| 19 | 20% | 1.50% | 1.50% | Smelinite | 2 | 1.833 |
| 20 | 20% | 2.50% | 2.50% | Smelinite | 1.7 | 1.583 |
| 21 | 20% | 1.50% | 1.50% | Kadox | 1.6 | 1.583 |
| | | | | 911C | | |
| 22 | 20% | 2.50% | 2.50% | Kadox | 1.7 | 1.666 |
| | | | | 911C | | |

TABLE S

| | | | | | SAE J1351 | SAE J1351 |
|---|---|---|---|---|---|---|
| | | | | | Odor | Odor |
| | Polydyne | Alterin | | | Rating | Rating |
| Formulation: | 80 | 110 | % PCA | PCA | 5 members | 6 members |
| 23 | 20% | 1% | 1% | Dixie Clay | 2.6 | 2.416 |
| | | | 1% | HC400 | | |
| 24 | 20% | 1% | 1% | McNamee Clay | 2.4 | 2.25 |
| | | | 1% | HC400 | | |
| 25 | 20% | 1% | 1% | Dixie Clay | 2.1 | 2 |
| | | | 1% | HC1400 | | |
| 26 | 20% | 1% | 1% | McNamee Clay | 2.3 | 2.1667 |
| | | | 1% | HC1400 | | |
| 27 | 20% | 1% | 1% | Dixie Clay | 1.9 | 1.75 |
| | | | 1% | HC2000 | | |
| 28 | 20% | 1% | 1% | McNamee Clay | 1.5 | 1.4167 |
| | | | 1% | HC2000 | | |
| 29 | 20% | 1% | 1% | Dixie Clay | 1.5 | 1.5 |
| | | | 1% | HC2100 | | |
| 30 | 20% | 1% | 1% | McNamee Clay | 1.4 | 1.333 |
| | | | 1% | HC2100 | | |
| 31 | 20% | 1% | 1% | Dixie Clay | 2.1 | 1.9167 |
| | | | 1% | Kadox 911C | | |
| 32 | 20% | 1% | 1% | McNamee Clay | 1.7 | 1.5833 |
| | | | 1% | Kadox 911C | | |
| 33 | 20% | 3% | 1% | Dixie Clay | 1.7 | 1.833 |
| | | | 1% | HC400 | | |
| 34 | 20% | 3% | 1% | McNamee Clay | 1.7 | 1.5833 |
| | | | 1% | HC400 | | |
| 35 | 20% | 3% | 1% | Dixie Clay | 2 | 1.833 |
| | | | 1% | HC1400 | | |
| 36 | 20% | 3% | 1% | McNamee Clay | 2.4 | 2.25 |
| | | | 1% | HC1400 | | |
| 37 | 20% | 3% | 1% | Dixie Clay | 2.1 | 2 |
| | | | 1% | HC2000 | | |
| 38 | 20% | 3% | 1% | McNamee Clay | 1.7 | 1.5833 |
| | | | 1% | HC2000 | | |
| 39 | 20% | 3% | 1% | Dixie Clay | 2.1 | 2 |
| | | | 1% | HC2100 | | |
| 40 | 20% | 3% | 1% | McNamee Clay | 2 | 1.833 |
| | | | 1% | HC2100 | | |
| 41 | 20% | 3% | 1% | Dixie Clay | 1.8 | 1.6667 |
| | | | 1% | Kadox 911C | | |
| 42 | 20% | 3% | 1% | McNamee Clay | 1.9 | 1.8333 |
| | | | 1% | Kadox 911C | | |
| 43 | 20% | 3% | 1% | HC400 | 1.7 | 1.5833 |
| | | | 1% | Kadox 911C | | |

TABLE T

| | | | | | SAE J1351 | SAE J1351 |
|---|---|---|---|---|---|---|
| | | | | | Odor | Odor |
| | Polydyne | Alterin | | | Rating | Rating |
| Formulation: | 80 | 110 | % PCA | PCA | 5 members | 6 members |
| Ctrl. Carrier | None | None | None | | 2.3 | 2.16 |
| 1 extruded | None | None | None | | 2.3 | 2.08 |
| Ctrl. Carrier | 20% | None | None | | 3.8 | 3.66 |
| 44 | 20% | 3% | 3% | Dixie Clay | 2.5 | 2.33 |
| 45 | 20% | 3% | 2% | Dixie Clay | 2.4 | 2.25 |
| | | | 1% | McNamee Clay | | |
| 46 | 20% | 3% | 2% | Dixie Clay | 2.2 | 2 |
| | | | 1% | HC 2100 | | |
| 47 | 20% | 3% | 2% | Dixie Clay | 2.45 | 2.21 |
| | | | 1% | Kadox 911C | | |
| 48 | 20% | 1.50% | 1.50% | Dixie Clay | 2.2 | 2.1 |
| | | | 1.50% | HC 2100 | | |
| | | | 1.50% | Kadox 911C | | |
| 49 | 20% | 1.50% | 1.50% | Dixie Clay | 2.2 | 2 |
| | | | 1.50% | HC 400 | | |
| | | | 1.50% | Kadox 911C | | |
| 50 | 20% | | 5% | HC 400 | 2.1 | 2 |
| 51 | 20% | | 6% | HC 400 | 2.2 | 2.08 |
| 52 | 20% | | 5% | HC 2100 | 2.2 | 2.16 |
| 53 | 20% | | 6% | HC 2100 | 2.2 | 2.08 |
| 54 | 20% | 3% | 3% | Kadox 911C | 2.3 | 2.08 |
| 55 | 20% | 2% | 2% | Dixie Clay | 2.6 | 2.5 |
| | | | 2% | HC 400 | | |
| 56 | 20% | 2% | 2% | Dixie Clay | 2.2 | 2.08 |
| | | | 2% | HC 2100 | | |
| 57 | 20% | 4% | 2% | Dixie Clay | 2.6 | 2.33 |
| 58 | 20% | 4% | 2% | HC 2100 | 2.1 | 1.92 |
| 59 | 20% | 4% | 2% | Kadox 911C | 2.3 | 2.08 |

TABLE U

| | | | | | SAE J1351 | SAE J1351 |
|---|---|---|---|---|---|---|
| | | | | | Odor | Odor |
| | Polydyne | Alterin | | | Rating | Rating |
| Formulation: | 80 | 110 | % PCA | PCA | 5 members | 6 members |
| 60 | 20% | 2.50% | 2.50% | Dixie Clay | 1.8 | 1.75 |
| | | | 1% | HC 400 | | |
| 61 | 20% | 2.50% | 2.50% | Dixie Clay | 1.9 | 1.83 |
| | | | 1% | HC 2100 | | |
| 62 | 20% | 2.50% | 2.5 | Dixie Clay | 2.1 | 1.92 |
| | | | 1% | Kadox 911C | | |
| 63 | 20% | 3.00% | 3% | Dixie Clay | 1.9 | 1.83 |
| | | | 1% | HC 400 | | |
| 64 | 20% | 3% | 3% | Dixie Clay | 1.8 | 1.75 |
| | | | 1% | HC 2100 | | |
| 65 | 20% | 3% | 3% | Dixie Clay | 1.6 | 1.58 |
| | | | 1% | Kadox 911C | | |
| 66 | 20% | 2.00% | 4% | Dixie Clay | 1.6 | 1.5 |
| | | | 1% | HC 2100 | | |
| 67 | 20% | 3.00% | 3% | McNamee Clay | 2 | 1.92 |
| | | | 1% | HC 2100 | | |
| 68 | 20% | 2% | 3% | McNamee Clay | 1.7 | 1.58 |
| | | | 1% | HC 2100 | | |
| 69 | 20% | | 5% | Dixie Clay | 2.4 | 2.3 |
| 70 | 20% | | 5% | McNamee Clay | 2.1 | 2.08 |
| 71 | 20% | | 2.50% | Dixie Clay | 1.9 | 1.83 |
| | | | 2.50% | HC 2100 | | |
| 72 | 20% | | 2.50% | McNamee Clay | 2.3 | 2.1 |
| | | | 2.50% | HC 2100 | | |
| 73 | 20% | 2.50% | 2.50% | Bentone 108 | 1.8 | 1.66 |

TABLE V

| | | | | | SAE J1351 | SAE J1351 |
|---|---|---|---|---|---|---|
| | | | | | Odor | Odor |
| | Polydyne | Alterin | | | Rating | Rating |
| Formulation: | 80 | 110 | % PCA | PCA | 5 members | 6 members |
| 74 | 20% | 1.50% | 1.50% | HC 2100 | 2.3 | 2.167 |
| | | | 1.50% | Bentone 108 | | |
| 75 | 20% | | 3% | Bentone 108 | 2 | 1.88 |
| 76 | 20% | | 5% | Bentone 108 | 2.2 | 2.05 |
| 77 | 20% | 2% | 2% | HC 2100 | 2.4 | 2.16 |
| | | | 1% | Bentone 108 | | |
| 78 | 20% | 3% | 1% | HC 2100 | 2.6 | 2.416 |
| | | | 1% | Bentone 108 | | |
| 79 | 20% | 2% | 1% | McNamee Clay | 2.1 | 1.93 |
| | | | 1% | HC 2100 | | |
| | | | 1% | Bentone 108 | | |
| 80 | 20% | 2% | 2% | McNamee Clay | 1.9 | 1.8 |
| | | | 1% | Bentone 108 | | |
| 81 | 20% | 3% | 3% | McNamee Clay | 2.2 | 2.08 |
| 82 | 20% | 2% | 2% | McNamee Clay | 2.2 | 2.08 |
| | | | 2% | HC 2100 | | |
| 83 | 20% | 3% | 3% | HC 2100 | 2.3 | 2.166 |
| 84 | 20% | 3% | 1% | McNamee Clay | 2.2 | 2.08 |
| | | | 1% | HC 2100 | | |
| | | | 1% | Kadox 911C | | |
| 85 | 20% | 2% | 1.50% | McNamee Clay | 2.3 | 2.166 |
| | | | 1.50% | HC 2100 | | |
| | | | 1.00% | Kadox 911C | | |
| 86 | 20% | 5% | 5% | McNamee Clay | 1.6 | 1.5 |
| 87 | 20% | 5% | 5% | HC 2100 | 2.1 | 1.95 |
| 88 | 20% | 5% | 5% | Bentone 108 | 1.9 | 1.78 |
| 89 | 20% | 5% | 5% | Dixie Clay | 2.1 | 1.95 |
| 90 | 20% | 4% | 2% | McNamee Clay | 2 | 1.83 |
| | | | 2% | HC 2100 | | |
| 91 | 20% | 4% | 4% | McNamee Clay | 3.8 | 3.716 |
| | | | 2% | HC 2100 | | |

The samples in this test were run on a 21 mm twin screw extruder, at 378 to 380 degrees F. or 192 to 193 degrees C. processing temperatures. The five member odor panel was made up of three men and two women, for the six member panel, an additional male nonsmoker was added. The samples were stored of 30 days in the dark prior to treatment in the oven for one hour at 65 degrees C. The sample still had residual moisture after 20 days storage. Due to the residual moisture on the pellets, all samples processed at 65 degrees C. for one hour give off lots of steam when the jar is opened for the odor panel to sniff. This moisture combines with the volatile organic compounds coming off the sample that add a musty or moldy component to the odor. It is believed that this tends to bias the ratings to a higher number than they would have if the samples were dry. Note: SAE J1351 calls for two type odor panels. One with dry pellets the other with added moisture on the pellets. The later method with moisture provides for higher overall odor ratings than dry pellets.

In a separate test, a polypropylene (PP) homopolymer, having stabilization additives of 0.1% by weight BHT and 0.1% by weight zinc stearate, was extruded without the addition of any functional additive. To this base homopolymer a functional additive was added, at the rate of 10% and 20%, without the addition of any odor control functional additives. The functional additive was PolyDyne™ available from Lehigh Technologies, Inc., Naples, Fla. and is made from recycled tires. This Polydyne™ functional additive has a small amount of odors from a plurality of organolepic sources. Control samples and samples containing odor control agents were prepared. Each sample was evaluated by a panel of six individuals in accordance with the standardized odor test SAE-J1351. The rating is an average of the scores given by the individuals on the panel.

The odor control agents that were used in the test are classified as follows: Alterin 110 is a clinoptilolite; Dixie Clay is a soft clay; McNamee is a hard clay; HC2100 is a nepheline syenite; Minex is a combination of silicon dioxide, aluminum oxide, sodium oxide along with minor amounts of some other metal oxides, the Minex 10 has mean particle size of 45 microns, the Minex 2 has a mean particle size of 106 microns; the 1806, 1709, 11132 are all silica gels where 1806 have beads ranging in size from 0.5 to 2 mm, 11132 is in a granular form, where the granules range in size from 0.7-1.4 mm.

TABLE 1
Odor Tested in Accordance with SAE-1351

| | | | | | % |
|---|---|---|---|---|---|
| Sample | % Polydyne | OCA | % OCA | Odor Rating | reduction |
| A | 0 | NA | NA | 1.333 | NA |
| B | 10 | NA | NA | 3.5833 | NA |
| C | 20 | NA | NA | 2.9166 | NA |
| D | 20 | Alterin 110 | 5% | 2.4166 | 17% |
| E | 20 | Alterin 110 | 10% | 2.1666 | 26% |
| F | 20 | Alterin 110 | 15% | 2 | 31% |
| G | 20 | Dixie Clay | 5% | 2.1666 | 26% |
| H | 20 | McNamee | 5% | 2.0833 | 29% |
| I | 20 | HC2100 | 5% | 2.0833 | 29% |
| J | 20 | Minex 10 | 5% | 2.0833 | 29% |
| K | 20 | Minex 2 | 5% | 1.9166 | 34% |
| L | 20 | Sil 1806 | 5% | 2.75 | 6% |
| M | 20 | Sil 11132 | 5% | 2.75 | 6% |
| N | 20 | Sil 1709 | 5% | 2.1666 | 26% |
| O | 20 | Alterin 110 | 5% | | |
| | | McNamee | 5% | 2.6666 | 9% |
| P | 20 | Alterin 110 | 5% | | |
| | | HC2100 | 5% | 2.583 | 11% |
| Q | 20 | Alterin 110 | 5% | | |
| | | Minex 2 | 5% | 2.4166 | 17% |
| R | 20 | Alterin 110 | 5% | | |
| | | Sil 1806 | 5% | 2.5 | 14% |
| S | 20 | Alterin 110 | 5% | | |
| | | Sil 11132 | 5% | 2.3333 | 20% |
| T | 20 | Alterin 110 | 5% | | |
| | | Sil 1709 | 5% | 1.75 | 40% |
| U | 20 | Alterin 110 | 7% | | |
| | | Sil 1806 | 7% | 2.6666 | 9% |
| V | 20 | Alterin 110 | 7% | | |
| | | Sil 11132 | 7% | 1.9166 | 34% |
| W | 20 | Alterin 110 | 7% | | |
| | | Sil 1709 | 7% | 1.6666 | 43% |

In order to try and determine what was happening chemically when one looks at the odor readings after the addition of the odor control agent it was decided to make up several samples and have them tested using gas chromatography-mass spectrometry (GC-MS). Gas chromatography-mass spectrometry (GC-MS) is a method that combines the features of gas-liquid chromatography and mass spectrometry to identify different substances within a test sample. The GC-MS has been widely heralded as a “gold standard” for substance identification because it is used to perform a specific test. A specific test positively identifies the actual presence of a particular substance in a given sample.

A test was performed using a virgin polypropylene resin. A control reading was taken off the polypropylene by itself and the polypropylene at a level of 10% and 20% Polydyne 80. Then different quantities of the odor control agent were added to the samples at the 20% Polydyne 80 level. It was found that 3 different gases were being given off by samples these were chloroform, various ketones, and branched hydrocarbons. When the odor control agent was added the total levels of vapor was significantly reduced but it was also detected that a fourth exhaust gas formed which was quinoline.

The odor control agents that were used in the test are classified as follows: Alterin 110 is a clinoptilolite; Dixie Clay is a soft clay; McNamee is a hard clay; HC2100 is a nepheline syenite; Minex is a combination of silicon dioxide, aluminum oxide, sodium oxide along with minor amounts of some other metal oxides, the Minex 10 has mean particle size of 45 microns, the Minex 2 has a mean particle size of 106 microns; the 1806, 1709, 11132 are all silica gels where 1806 have beads ranging in size from 0.5 to 2 mm, 11132 is in a granular form, where the granules range in size from 0.7-1.4 mm.

TABLE 2
GC/MS Odor Control Agents all at 20% Polydyne

| | Inorganics | Total Response Load | Odor Rating |
|---|---|---|---|
| | & Levels | (GC/MS Components) | (Subjective) |
| PP 1 | Control | 270405018 | 1.333 |
| 2 | 10% PD80 | 341152679 | 3.583 |
| 3 | 20% PD80 | 364948189 | 2.917 |
| 4 | 5% Alterin 110 | 6968832 | 2.417 |
| 5 | 10% Alterin 110 | 33821265 | 2.167 |
| 5B | 15% Alterin 110 | 35107907 | 2 |
| 6 | 5% Dixie Clay | 29698452 | 2.167 |
| 7 | 5% McNamee | 40184134 | 2.083 |
| 8 | 5% HC2100 | 52347288 | 2.083 |
| 9 | 5% Minex 10 | 12061626 | 2.083 |
| 10 | 5% Minex 2 | 5358705 | 1.9166 |
| 14 | 5% ea.110: McNamee | 13602346 | 2.666 |
| 15 | 5% ea.110: HC2100 | 43381959 | 2.583 |
| 16 | 5% ea.110: Minex 2 | 38256110 | 2.416 |
| 17 | 5% ea.110: 1806 silica | 40282929 | 2.5 |

TABLE 3
GC/MS showing level of Chloroform

| | | | | From Col.D |
|---|---|---|---|---|
| | | | | Percent |
| | | | | Chloroform |
| | | Odor Rating | | Reduction |
| | Inorganics & Levels | (Subjective) | Chloroform | vs.#3 |
| PP 1 | Control | 1.333 | 264413379 | |
| 2 | 10% PD80 | 3.583 | 335723237 | |
| 3 | 20% PD80 | 2.917 | 359699657 | |
| 4 | 5% Alterin 110 | 2.417 | 3108300 | 99.14 |
| 5 | 10% Alterin 110 | 2.167 | 2227211 | 99.38 |
| 5B | 15% Alterin 110 | 2 | 1152450 | 99.68 |
| 6 | 5% Dixie Clay | 2.167 | 1830460 | 99.49 |
| 7 | 5% McNamee | 2.083 | 1233682 | 99.66 |
| 8 | 5% HC2100 | 2.083 | 12891497 | 96.42 |
| 9 | 5% Minex 10 | 2.083 | 1473853 | 99.59 |
| 10 | 5% Minex 2 | 1.9166 | 1666307 | 99.54 |
| 14 | 5% ea.110: | 2.666 | 1202157 | 99.67 |
| | McNamee | | | |
| 15 | 5% ea.110: HC2100 | 2.583 | 1318966 | 99.63 |
| 16 | 5% ea.110: Minex 2 | 2.416 | 1153311 | 99.68 |
| 17 | 5% ea.110: 1806 | 2.5 | 889794 | 99.75 |
| | silica | | | |

TABLE 4
GC/MS showing levels of Ketones

| | | | | From |
|---|---|---|---|---|
| | | | | Column D |
| | | | | Percent |
| | | | | Ketone |
| | | Odor Rating | | Reduction |
| | Inorganics & Levels | (Subjective) | Ketone | vs.#3 |
| PP 1 | Control | 1.333 | 2834937 | |
| 2 | 10% PD80 | 3.583 | 2240713 | |
| 3 | 20% PD80 | 2.917 | 2048446 | |
| 4 | 5% Alterin 110 | 2.417 | 1326452 | 35.25 |
| 5 | 10% Alterin 110 | 2.167 | 964465 | 52.92 |
| 5B | 15% Alterin 110 | 2 | 778118 | 62.01 |
| 6 | 5% Dixie Clay | 2.167 | 1022234 | 50.1 |
| 7 | 5% McNamee | 2.083 | 1023058 | 50.06 |
| 8 | 5% HC2100 | 2.083 | 1094806 | 46.55 |
| 9 | 5% Minex 10 | 2.083 | 1301811 | 36.45 |
| 10 | 5% Minex 2 | 1.9166 | 1156343 | 43.55 |
| 14 | 5% ea.110: McNamee | 2.666 | 1008880 | 50.75 |
| 15 | 5% ea.110: HC2100 | 2.583 | 1013326 | 50.53 |
| 16 | 5% ea.110: Minex 2 | 2.416 | 1182976 | 42.25 |
| 17 | 5% ea.110: 1806 silica | 2.5 | 959720 | 53.15 |

TABLE 5
GC/MS showing levels of Branched Hydrocarbons

| | | | | From Col.D |
|---|---|---|---|---|
| | | | | Percent |
| | | | Branched | Bran. Hydro. |
| | | Odor Rating | Hydro- | reduction |
| | Inorganics & Levels | (Subjective) | carbons | vs.#3 |
| PP 1 | Control | 1.333 | 3156702 | |
| 2 | 10% PD80 | 3.583 | 3188729 | |
| 3 | 20% PD80 | 2.917 | 3200086 | |
| 4 | 5% Alterin 110 | 2.417 | 2533980 | 20.82 |
| 5 | 10% Alterin 110 | 2.167 | 2336454 | 26.99 |
| 5B | 15% Alterin 110 | 2 | 2020008 | 36.88 |
| 6 | 5% Dixie Clay | 2.167 | 2431581 | 24.02 |
| 7 | 5% McNamee | 2.083 | 2457024 | 23.22 |
| 8 | 5% HC2100 | 2.083 | 2433923 | 23.94 |
| 9 | 5% Minex 10 | 2.083 | 2402686 | 24.92 |
| 10 | 5% Minex 2 | 1.9166 | 2536055 | 20.75 |
| 14 | 5% ea.110: McNamee | 2.666 | 2391309 | 25.27 |
| 15 | 5% ea.110: HC2100 | 2.583 | 2270578 | 29.05 |
| 16 | 5% ea.110: Minex 2 | 2.416 | 2212764 | 30.85 |
| 17 | 5% ea.110: 1806 silica | 2.5 | 2081011 | 34.97 |

TABLE 6
GC/MS showing levels of Quinolilnes

| | | | | From Col.D |
|---|---|---|---|---|
| | | | | Percent |
| | | | | Quinoline |
| | | Odor Rating | | Reduction |
| | Inorganics & Levels | (Subjective) | Quinolilne | vs. #3 |
| PP 1 | Control | 1.333 | None | |
| 2 | 10% PD80 | 3.583 | None | |
| 3 | 20% PD80 | 2.917 | None | |
| 4 | 5% Alterin 110 | 2.417 | None | None |
| 5 | 10% Alterin 110 | 2.167 | 28293135 | More |
| 5B | 15% Alterin 110 | 2 | 31157331 | More |
| 6 | 5% Dixie Clay | 2.167 | 24414177 | More |
| 7 | 5% McNamee | 2.083 | 35470370 | More |
| 8 | 5% HC2100 | 2.083 | 35027062 | More |
| 9 | 5% Minex 10 | 2.083 | 6883276 | More |
| 10 | 5% Minex 2 | 1.9166 | None | None |
| 14 | 5% ea.110: McNamee | 2.666 | None | None |
| 15 | 5% ea.110: HC2100 | 2.583 | 38779089 | More |
| 16 | 5% ea.110: Minex 2 | 2.416 | 33707059 | More |
| 17 | 5% ea.110: 1806 silica | 2.5 | 36352404 | More |

A test was performed using a virgin polypropylene resin and the functional additive at a level of 40% Polydyne 80. Then different quantities of the odor control agent were added to the samples at the 40% Polydyne 80 level. It was found that 6 different gases were being given off by these samples. The gases are branched hydrocarbons, n-cyclohexyl cyclohexamine (CAS 101-83-7) referred to in the tables as component 1, benzothlazole (CAS 95-16-9) referred to in the tables as component 2,2-methyl benzaldehyde (CAS 629-20-4) referred to in the tables as component 3, n-(2,2-dimethylpropyl)-n-methyl aniline (CAS 53927-81-0) referred to in the tables as component 4, and 2,4-di-tert butyl phenol (CAS 96-76-3) referred to in the tables as component 5.

The odor control agents that were used in the test are classified as follows: Alterin 110 is a clinoptilolite; Dixie Clay is a soft clay; MuG 400 is a clay; the Absents are synthetic zeolites available from UOP LLC of Des Plaines, Ill. Wax 1602 and wax 2602 are metallocene waxes available from Clariant Corporation, Charlotte N.C.

TABLE 7
GC/MS 40% Polydyne showing levels of total emissions

| | | | | Total Load of |
|---|---|---|---|---|
| | | | 65 C./1 hr | GC/MS |
| Tested | | 65 C./1 hr | Odor | Detector |
| GCMS | | Odor | Rate | (All five |
| Sample # | | Rating | Avg | components) |
| 1 Ctrl | None | | | 1,980,293 |
| 1 oven | None | 2.3 | 2.3 | 2,018,978 |
| treated | | | | |
| 2 | 10% Alterin 110 | 2.7 | 2.7 | 1,706,349 |
| 3 | 20% Alterin 110 | 2.1 | 2.1 | 1,706,147 |
| 6 | 10% Dixie Clay | 2.2 | 2.2 | 2,191,032 |
| 10 | 10% Absent 1000 | 2.4 | 2.4 | 1,056,768 |
| | 5% Alterin 110 | | | |
| 13 | 5% Absent 2000 | 2.1 | 2.1 | 994,720 |
| | 10% MuG400 Clay | | | |
| 18 | 10% Alterin 110 | 2 | 2 | 1,635,277 |
| | 5% Absent 3000 | | | |
| 46 | 5% Wax 1602 | 1.97-2.1 | 2 | 599,837 |
| | 10% Alterin 110 | | | |
| | 5% Absent 3000 | | | |
| 48 | 10% Wax 2602 | 1.73-1.88 | 1.79 | 556,328 |
| | 10% Alterin 110 | | | |
| | 5% Absent 2000 | | | |
| 50 | 5% Absent 3000 | 1.9-2.1 | 2 | 316,080 |
| | 10% Alterin 110 | | | |
| | 5% Absent 1000 | | | |
| 51 | 5% Absent 3000 | 1.75-1.9 | 1.8 | 338,372 |

TABLE 8
GC/MS at 40% Polydyne showing levels Branched Hydrocarbons

| | | C9-C16 | % |
|---|---|---|---|
| Tested | | Branched | Reduction |
| GCMS | | Oils | Branched |
| Sample # | | Hydrocarbons | oils |
| 1 Ctrl | None | 1453530 | |
| 1 oven | None | 1510088 | |
| treated | | | |
| 2 | 10% Alterin 110 | 1311505 | 10% |
| 3 | 20% Alterin 110 | 822296 | 50% |
| 6 | 10% Dixie Clay | 1702524 | 0 |
| 10 | 10% Absent 1000 | 996983 | 30% |
| | 5% Alterin 110 | | |
| 13 | 5% Absent 2000 | 830108 | 50% |
| | 10% MuG400 | | |
| | Clay | | |
| 18 | 10% Alterin 110 | 1330571 | 10% |
| | 5% Absent 3000 | | |
| 46 | 5% Wax 1602 | 373081 | 80% |
| | 10% Alterin 110 | | |
| | 5% Absent 3000 | | |
| 48 | 10% Wax 2602 | 315323 | 80% |
| | 10% Alterin 110 | | |
| | 5% Absent 2000 | | |
| 50 | 5% Absent 3000 | 268640 | 80% |
| | 10% Alterin 110 | | |
| | 5% Absent 1000 | | |
| 51 | 5% Absent 3000 | 386030 | 70% |

TABLE 9
GC/MS at 40% Polydyne showing levels of Component 1

| | | | Relative to #1 oven |
|---|---|---|---|
| Tested | | | treated |
| GCMS | | | % Reduction of |
| Sample # | | Component 1 | Component 1 |
| 1 Ctrl | None | 155498 | |
| 1 oven | None | 150445 | |
| treated | | | |
| 2 | 10% Alterin 110 | 74848 | 50% |
| 3 | 20% Alterin 110 | 25504 | 80% |
| 6 | 10% Dixie Clay | 107960 | 30% |
| 10 | 10% Absent 1000 | 2461 | 99% |
| | 5% Alterin 110 | | |
| 13 | 5% Absent 2000 | 28739 | 80% |
| | 10% MuG400 | | |
| | Clay | | |
| 18 | 10% Alterin 110 | 31180 | 80% |
| | 5% Absent 3000 | | |
| 46 | 5% Wax 1602 | 48929 | 70% |
| | 10% Alterin 110 | | |
| | 5% Absent 3000 | | |
| 48 | 10% Wax 2602 | 54329 | 60% |
| | 10% Alterin 110 | | |
| | 5% Absent 2000 | | |
| 50 | 5% Absent 3000 | 5793 | 99% |
| | 10% Alterin 110 | | |
| | 5% Absent 1000 | | |
| 51 | 5% Absent 3000 | 2352 | 99% |

TABLE 10
GC/MS at 40% Polydyne showing levels of Component 2

| | | | Relative to #1 oven |
|---|---|---|---|
| Tested | | | treated |
| GCMS | | | % Reduction of |
| Sample # | | Component 2 | Component 2 |
| 1 Ctrl | None | 350863 | |
| 1 oven | None | 343628 | |
| treated | | | |
| 2 | 10% Alterin 110 | 304068 | 10% |
| 3 | 20% Alterin 110 | 237429 | 30% |
| 6 | 10% Dixie Clay | 362685 | 0 |
| 10 | 10% Absent 1000 | 51061 | 90% |
| | 5% Alterin 110 | | |
| 13 | 5% Absent 2000 | 122771 | 60% |
| | 10% MuG400 | | |
| | Clay | | |
| 18 | 10% Alterin 110 | 265254 | 20% |
| | 5% Absent 3000 | | |
| 46 | 5% Wax 1602 | 164066 | 50% |
| | 10% Alterin 110 | | |
| | 5% Absent 3000 | | |
| 48 | 10% Wax 2602 | 172484 | 50% |
| | 10% Alterin 110 | | |
| | 5% Absent 2000 | | |
| 50 | 5% Absent 3000 | 34890 | 90% |
| | 10% Alterin 110 | | |
| | 5% Absent 1000 | | |
| 51 | 5% Absent 3000 | 43760 | 90% |

TABLE 11
GC/MS at 40% Polydyne showing levels of Component 3

| | | | Relative to #1 oven |
|---|---|---|---|
| Tested | | | treated |
| GCMS | | | % Reduction of |
| Sample # | | Component 3 | Component 3 |
| 1 Ctrl | None | 5274 | |
| 1 oven | None | 4644 | |
| treated | | | |
| 2 | 10% Alterin 110 | 3608 | 20% |
| 3 | 20% Alterin 110 | 0 | 100% |
| 6 | 10% Dixie Clay | 5859 | 0 |
| 10 | 10% Absent 1000 | 1426 | 70% |
| | 5% Alterin 110 | | |
| 13 | 5% Absent 2000 | 2088 | 60% |
| | 10% MuG400 | | |
| | Clay | | |
| 18 | 10% Alterin 110 | 0 | 100% |
| | 5% Absent 3000 | | |
| 46 | 5% Wax 1602 | 4004 | 10% |
| | 10% Alterin 110 | | |
| | 5% Absent 3000 | | |
| 48 | 10% Wax 2602 | 4012 | 10% |
| | 10% Alterin 110 | | |
| | 5% Absent 2000 | | |
| 50 | 5% Absent 3000 | 0 | 100% |
| | 10% Alterin 110 | | |
| | 5% Absent 1000 | | |
| 51 | 5% Absent 3000 | 0 | 100% |

MuG 400 is available from Active Minerals International LLC, Hunt Valley Maryland and is an example of a Palygorskite or Attapulgite.

TABLE 12
GC/MS at 40% Polydyne showing levels of Component 4

| | | | Relative to #1 oven |
|---|---|---|---|
| Tested | | | treated |
| GCMS | | | % Reduction of |
| Sample # | | Component 4 | Component 4 |
| 1 Ctrl | none | 10280 | |
| 1 oven | none | 10134 | |
| treated | | | |
| 2 | 10% Alterin 110 | 8404 | 17% |
| 3 | 20% Alterin 110 | 7724 | 24% |
| 6 | 10% Dixie Clay | 8722 | 10% |
| 10 | 10% Absent 1000 | 2863 | 70% |
| | 5% Alterin 110 | | |
| 13 | 5% Absent 2000 | 7582 | 30% |
| | 10% MuG400 | | |
| | Clay | | |
| 18 | 10% Alterin 110 | 6091 | 40% |
| | 5% Absent 3000 | | |
| 46 | 5% Wax 1602 | 7787 | 23% |
| | 10% Alterin 110 | | |
| | 5% Absent 3000 | | |
| 48 | 10% Wax 2602 | 7935 | 22% |
| | 10% Alterin 110 | | |
| | 5% Absent 2000 | | |
| 50 | 5% Absent 3000 | 5324 | 50% |
| | 10% Alterin 110 | | |
| | 5% Absent 1000 | | |
| 51 | 5% Absent 3000 | 4478 | 60% |

TABLE 13
GC/MS at 40% Polydyne showing levels of Component 5

| Tested | | | |
|---|---|---|---|
| GCMS | | | % Reduction of |
| Sample # | | Component 5 | Component 5 |
| 1 Ctrl | none | 4848 | |
| 1 oven treated | none | 3903 | |
| 2 | 10% Alterin 110 | 3718 | 5% |
| 3 | 20% Alterin 110 | 1787 | 54% |
| 6 | 10% Dixie Clay | 3282 | 16% |
| 10 | 10% Absent | 1954 | 50% |
| | 1000 | | |
| | 5% Alterin 110 | | |
| 13 | 5% Absent 2000 | 3432 | 12% |
| | 10% MuG400 | | |
| | Clay | | |
| 18 | 10% Alterin 110 | 2181 | 44% |
| | 5% Absent 3000 | | |
| 46 | 5% Wax 1602 | 1970 | 50% |
| | 10% Alterin 110 | | |
| | 5% Absent 3000 | | |
| 48 | 10% Wax 2602 | 2245 | 43% |
| | 10% Alterin 110 | | |
| | 5% Absent 2000 | | |
| 50 | 5% Absent 3000 | 1233 | 68% |
| | 10% Alterin 110 | | |
| | 5% Absent 1000 | | |
| 51 | 5% Absent 3000 | 1752 | 55% |

A sample of neat Polydyne 80 alone, without the plastic resin, was tested using pyrolysis gas chromatography-mass spectrometry (GC-MS). The sample was heated to 225 degrees C and this pyrolysis gave off molecules such as: Acetaldehyde, Isobutene, Sulfur dioxide, Aminomethanesulfonic acid, Ethanol, Acetone, 2-Propanamine, 2-methyl-, Carbon disulfide, 1-Pentene, 4methyl-, 1-Butene, 2,3-dimethyl-, Cyclopropane. 1,1,2-trimethyl-, Acetic acid, Furan, 2-methyl Diisopropylamine, Piperazine, Propanoic acid, Cyclobutane, ethenyl-, Cyclohexene, Methyl Isobutyl Ketone, 2-Pentanamine, 4-methyl-, R-(−)-Cyclohexylethylamine, 2-Hexanamine, 4-methyl-, Toluene, Morpholine, Cyclopentane, 1,2,4-trimethyl-, 2-Heptene, 3-methyl-, (3-Aminopropyl)dipropylborane, Benzene, 1,3-dimethyl-, p-Xylene, Benzene, 1,2-dimethyl-, Cyclohexene, Cyclopropane, 1,2-dimethyl-, ciscyclohexanamine, Cyclopentanamine, Pyridine, 3-methyl-, Aniline, Cyclohexane, isocyanato-, 1,1′-Bicyclopropyl, Cyclobutane, ethenyl-, Hexanoic acid, 2-ethyl-, 1,4-Dioxane, 2,4-Nonadiene, (E,E)-, 1,2-Dipentylcyclopropene, 2,3-Nonadiene, various alkyl butanoic acids, pentanoic acids, hexanoic acids and neodecanoic acids, 2-Methyl-3-(methylthio)-1-propene, Benzothiazole, Nonane, 5-(2-methylpropyl)-, various alkanes and alkenes. From this it is believed that when the Polydyne is added to a resin and processed that the organic compounds, which come off from the composite, may be different, however the addition of the odor control agent helps to significantly reduce the type and levels volatile organic compounds, which might be volatized and extracted.

What is claimed is:

1. A method for capturing organoleptic odors and volatile and semi-volatile organic compounds in a mixture of a resin and a functional additive comprising the steps of:
providing a plastic resin;
providing a functional additive where said functional additive has an odor from a plurality of organolepic sources;
blending said functional additive at a level of from 2% by weight to 70% by weight of said plastic resin, with said plastic resin;
blending in an odor control agent at a level from 1% to 30% by weight of said resin, where said odor control agent is selected from the group consisting of: nepheline syenite, nepheline, silica gel, hydrogels, hard and soft clays, hydrous magnesiuim aluminum silicates, bentonite, clinoptilolite (Bulgarian) from both sodium and potassium clinoptilolite forms, hectorite, cationic exchanged clinoptilolites (with silver, copper, nickel), cerium, cesium and other cation elements exchanged within the cage structure, chabazite, faujasite, gmelinite, brewsterite, calcium meta silicate, calcium silicate, magnesium aluminum hydroxy carbonates, zinc oxide, zinc hydroxide, zinc carbonate, calcium oxide, calcium hydroxide, calcium carbonate, potassium meta phosphate, silver oxide, magnesium hydroxide, magnesium oxide, copper oxide, ferric and ferrous oxides, sorbitol, glucitol, mannitol, glucose, dextrose, dextrin, allophanes, structured silica, amorphous silicas, sodalite,hydrotalcites (natural and synthetic); silicon oxide and dioxide, aluminum oxide and dioxides, natural zeolites, synthetic zeolites, ammonia treated and acid treated clinoptilolite, metallocene waxes, manganese dioxide, nano zinc oxide and nano titanium and combination thereof, to produce a blended resin;
where said blended resin exudes volatile organic compounds; and
where said blended resin contains said odor control agent and has a reduction in volatile organic compounds exuded resulting in a reduction of odor as compared to a plastic resin and functional additive with no odor control agent added.

2. The method for chemically capturing organoleptic odor according to claim 1 where said functional additive is a cryogenically ground tire rubber or a ground tire rubber.

3. The method for chemically capturing organoleptic odor according to claim 1 where said blended resin exhibits at least a 5% reduction in odor based on a standardized odor test SAE-J1351.

4. The method for chemically capturing organoleptic odor according to claim 1 where said resin has a melting point from 115 to 250° C.

5. The method for chemically capturing organoleptic odor according to claim 4 where said resin is selected from the group comprising: polypropylene, polyethylene, polybutylene, a copolymer of acrylic, butadiene, cured and uncured EPDM rubbers, styrene (ABS), acetal, acrylic, nylons, phenylene oxide, polycarbonate, polyester, polysulfone, styrene, urethane, polyurethane, vinyl and combinations thereof.

6. The method for chemically capturing organoleptic odor according to claim 1 where said odor control agent is a blend of two or more odor control agents selected from the group of: nepheline syenite, nepheline, silica gel, clinoptilolite, magnesium aluminum silicate, natural zeolite and synthetic zeolite.

7. The method for chemically capturing organoleptic odor according to claim 6 where said two odor control agents are added in a ratio of 5:1 to 1:5.

8. The method for chemically capturing organoleptic odor according to claim 2 where said functional additive is added at a level of from 5% to 45% by weight of said plastic resin and said level of said odor control agent is from 5% to said 25% by weight of said plastic resin.

9. The method for chemically capturing organoleptic odor according to claim 1 where said odor control agent is a magnesium aluminum phyllosilicate.

10. A method for capturing organoleptic odor in a resin comprising the steps of:
providing a resin where said resin is selected from the group comprising: polypropylene, polyethylene, polybutylene, a copolymer of acrylic, butadiene, cured and uncured EPDM rubbers, styrene (ABS), acetal, acrylic, nylon. phenylene oxide, polycarbonate, polyester, polysulfone, styrene, urethane, polyurethane, vinyl and combinations thereof;
blending in an odor control agent at a level from 1% to 30% by weight of said resin, where said odor control agent is selected from the group consisting of: nepheline syenite, nepheline, silica gel, hydrogels, hard and soft clays, bentonite, hydrous magnesium aluminum silicates, clinoptilolite (Bulgarian) from both sodium and potassium clinoptilolite forms, hectorite, cationic exchanged clinoptilolites (with silver, copper, nickel), cerium, cesium and other cation elements exchanged within the cage structure, chabazite, faujasite, gmelinite, brewsterite, calcium meta silicate, calcium silicate, magnesium aluminum hydroxy carbonates, zinc oxide, zinc hydroxide, zinc carbonate, calcium oxide, calcium hydroxide, calcium carbonate, potassium meta phosphate, silver oxide, magnesium hydroxide, magnesium oxide, copper oxide, ferric and ferrous oxides, sorbitol, glucitol, mannitol, glucose, dextrose, dextrin, allophanes, structured silica, amorphous silicas, sodalite, silicon oxide and dioxide, aluminum oxide and dioxides, natural zeolites, synthetic zeolites, ammonia treated and acid treated clinoptilolite, manganese dioxide, metallocene waxes, nano zinc oxide and nano titanium and combination thereof, to produce a blended resin;
where said blended resin exudes volatile organic compounds; and
where said blended resin containing said odor control agent and has a reduction in volatile organic compounds exuded resulting in a reduction of odor as compared to a plastic resin with no odor control agent added.

11. The method for chemically capturing organoleptic odor according to claim 10 where said resin has an odor produced from volatile organic compounds added to said resin.

12. The method for chemically capturing organoleptic odor according to claim 10 where said blended resin exhibits at least a 5% reduction in odor based on a standardized odor test SAE-J1351.

13. The method for chemically capturing organoleptic odor according to claim 10 where a magnesium aluminum silicate is blended with an odor control agents selected from the group of: nepheline syenite, nepheline, silica gel, clinoptilolite, natural zeolite and synthetic zeolite.

14. The method for chemically capturing organoleptic odor according to claim 10 further comprising the steps of:
providing a functional additive where said functional additive has an odor from a plurality of organolepic sources; and
blending said functional additive at a level of from 2% by weight to 70% by weight of said resin, with plastic resin.

15. The method for chemically capturing organoleptic odor according to claim 10 where said resin has a melting point from 115 to 250° C.

16. The method for chemically capturing organoleptic odor according to claim 14 where said functional additive is derived from recycled tires.

17. The method for chemically capturing organoleptic odor according to claim 10 where said odor control agent is a blend of two or more odor control agents selected from the group of: nepheline syenite, nepheline, silica gel, clinoptilolite, magnesium aluminum silicate, natural zeolite and synthetic zeolite.

18. The method for chemically capturing organoleptic odor according to claim 17 where said two odor control agents are added in a ratio of 5:1 to 1:5.

19. The method for chemically capturing organoleptic odor according to claim 14 where said functional additive is added at a level of from 5% to 45% by weight of said plastic resin and said level of said odor control agent is from 5% to said 25% by weight of said plastic resin.

20. A composition of matter comprising:
a resin where said resin is selected from the group comprising: polypropylene, polyethylene, polybutylene, a copolymer of acrylic, butadiene, and styrene (ABS); acetal; acrylic, nylon, phenylene oxide, polycarbonate, polyester, polysulfone, styrene, urethane, polyurethane, vinyl and combinations thereof;
a functional additive where said functional additive is cryogenically ground tire rubber at a level of from 2% to 70% by weight of said resin;
an odor control agent at a level from 1% to 30% by weight of said resin, where said odor control agent is selected from the group consisting of: nepheline syenite, nepheline, silica gel, hydrogels, hard and soft clays, hydrous magnesiuim aluminum silicates, bentonite, clinoptilolite (Bulgarian) from both sodium and potassium clinoptilolite forms, hectorite, cationic exchanged clinoptilolites (with silver, copper, nickel), cerium, cesium and other cation elements exchanged within the cage structure, chabazite, faujasite, gmelinite, brewsterite, calcium meta silicate, calcium silicate, magnesium aluminum hydroxy carbonates, zinc oxide, zinc hydroxide, zinc carbonate, calcium oxide, calcium hydroxide, calcium carbonate, potassium meta phosphate, silver oxide, magnesium hydroxide, magnesium oxide, copper oxide, ferric and ferrous oxides, sorbitol, glucitol, mannitol, glucose, dextrose, dextrin, allophanes, structured silica, amorphous silicas, sodalite, silicon oxide and dioxide, aluminum oxide and dioxides, natural zeolites, synthetic zeolites, ammonia treated and acid treated clinoptilolite, manganese dioxide, metallocene waxes, nano zinc oxide and nano titanium and combination thereof, to produce a blended resin.
